(19) United States
(12) Patent Application Publication
(10) Pub. No.: US 2023/0057788 A1
(43) Pub. Date: Feb. 23, 2023

(54) METHOD OF TREATING CHRONIC MYELOID LEUKEMIA USING PEGYLATED INTERFERON AND TYROSINE KINASE INHIBITOR

(71) Applicant: PharmaEssentia Corporation, Taipei (TW)

(72) Inventor: Ko-Chung Lin, Lexington, MA (US)

(21) Appl. No.: 17/882,087

(22) Filed: Aug. 5, 2022

Related U.S. Application Data
(60) Provisional application No. 63/229,851, filed on Aug. 5, 2021.

(51) Int. Cl. A61K 47/60; A61K 47/54; A61K 31/506; A61K 31/496; A61K 31/5025; A61P 35/02

(52) U.S. Cl. CPC A61K 47/60; A61K 47/545; A61K 31/506; A61K 31/496; A61K 31/5025; A61P 35/02

(57) ABSTRACT

Described herein is a method of treating chronic myeloid leukemia in a subject including administering to a subject in need thereof a pegylated interferon-α and a BCR-ABL tyrosine kinase inhibitor simultaneously or sequentially.

### RELATED APPLICATION

This application claims priority to U.S. Provisional Application No. 63/229,851, filed on Aug. 5, 2021, the content of which is hereby incorporated by reference herein.

### BACKGROUND

Chronic myeloid leukemia (CML) is a clonal stem-cell disorder characterized by the presence of the Philadelphia (Ph) chromosome. This reciprocal translocation between chromosomes 9 and 22 [t(9;22)(q34;q11)] results in a BCR-ABL1 fusion protein with a constitutively activated tyrosine kinase. See, Shtivelman et al., Nature (1985), 315:550-554; Lugo et al., Science (1990), 247(4946):1079-1082; and Chase et al., Best Pract Res Clin Haematol. (2001), 14(3):443-471.

The use of imatinib, an oral inhibitor targeting the ABL1 tyrosine kinase, has been approved for the treatment of pretreated and de novo CML since 2001 and 2003, respectively. Three other tyrosine kinase inhibitors, nilotinib, dasatinib, and bosutinib, have been approved for first-line treatment of CML. These agents achieve faster and deeper remissions. However, long term use of these drugs may be limited due to specific adverse events (AEs).

Interferon-α was introduced for the treatment of CML in the early 1980s and was recommended as a first-line treatment until 2001, when imatinib became available. Tolerability of interferons might be limited by various acute and chronic side effects.

### SUMMARY

In one aspect, described here is a method of treating chronic myeloid leukemia in a subject. The method includes administering to a subject in need thereof a pegylated interferon-α and a BCR-ABL tyrosine kinase inhibitor simultaneously or sequentially for a treatment period, wherein the pegylated interferon-α is a conjugate of formula I:

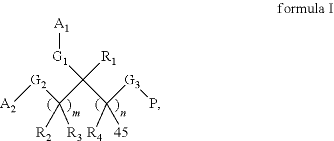

in which
each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;
each of A₁ and A₂, independently, is a polymer moiety;
each of G₁, G₂, and G₃, independently, is a bond or a linking functional group;
P is an interferon-α moiety;
m is 0 or an integer of 1-10; and
n is an integer of 1-10.

In some embodiments, the conjugate of formula I has one or more properties including: (i) a median Tmax in the range of 3 to 6 days following administration of multiple 50 to 540 μg doses of the conjugate once every two weeks to subjects; (ii) a mean T_{1/2} in the range of 6 to 10 days following administration of multiple 50 to 540 μg doses of the conjugate once every two weeks to subjects; and (iii) an individual maximum tolerated dose of at least 500 μg once every 2 to 4 weeks in subjects.

In some embodiments, the conjugate of formula I has one or more features including: G3 is a bond and P is an interferon-α moiety in which the amino group at the N-terminus is attached to G3; A₁ and A₂ are polyalkylene oxide moieties each having a molecular weight of 10-30 kD; each of G₁ and G₂ is

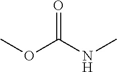

in which O is attached to A₁ or A₂, and NH is attached to a carbon atom as shown in formula I; each of R₁, R₂, R₃, R₄, and R₅ is H; m is 4 and n is 2; and the interferon-α moiety is a modified interferon-α moiety containing 1-4 additional amino acid residues.

In some embodiments, the interferon-α moiety is a human interferon-α_{2b} having a proline residue at the N-terminus and is 166 amino acids in length.

In some embodiments, conjugate of formula I is

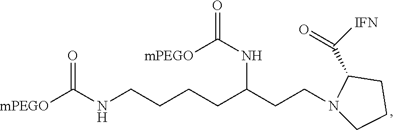

in which mPEG has a molecular weight of 20 kD and IFN is an interferon-α_{2b}.

In some embodiments, the tyrosine kinase inhibitor is imatinib, dasatinib, nilotinib, bosutinib, or ponatinib. For example, 100 to 800 mg of imatinib can be administered to the subject daily.

In some embodiments, 50 to 540 μg of the pegylated interferon-α is administered to the subject once every 2 to 8 weeks. In some embodiments, 50 to 100 μg of the pegylated interferon is administered to the subject once every 2 weeks.

In some embodiments, the treatment period is at least 6 months, at least 12 months, at least 18 months, at least 24 months, at least 30 months, at least 36 months, at least 42 months, at least 48 months, or at least 54 months or more.

In some embodiments, the subject is treated with the pegylated interferon-α and the BCR-ABL tyrosine kinase inhibitor sequentially. The subject can be first treated with the BCR-ABL tyrosine kinase inhibitor alone followed by the pegylated interferon-α alone, or can be first treated with the pegylated interferon-α alone followed by the BCR-ABL tyrosine kinase inhibitor alone.

In some embodiments, the subject has a reduction of BCR-ABL1 transcript to at least ≤0.01% or deeper, e.g., at least ≤0.0032% or deeper, at least ≤0.001% or deeper, or a non-detectable level, during or by end of the treatment period. The reduction can be detected at 12 months or earlier in the treatment period.

In some embodiments, the subject has a complete hematologic response or a cytogenetic response or both during or by end of the treatment period.

In some embodiments, the subject has less adverse events or lower grade adverse events than a subject treated with a combination of a BCR-ABL tyrosine kinase inhibitor and another pegylated interferon.

The details of one or more embodiments are set forth in the accompanying drawing and the description below. Other features, objects, and advantages of the embodiments will be apparent from the description and drawing, and from the claims.

### BRIEF DESCRIPTION OF DRAWINGS

FIG. 1 is a graph showing molecular responses in patients treated with a pegylated interferon-α and a BCR-ABL1 kinase inhibitor.

FIG. 2 is a graph showing course of BCR-ABL1 transcripts during a study period in which patients were treated with a pegylated interferon-α and a BCR-ABL1 kinase inhibitor.

### DETAILED DESCRIPTION

Described herein is a method of treating CML with a pegylated interferon-α and a BCR-ABL1 kinase inhibitor. The two compounds can be administered to a subject simultaneously or sequentially for a treatment period.

In a simultaneous treatment, multiple doses of a pegylated interferon-α and multiple doses of a BCR-ABL1 kinase inhibitor are administered together to a subject. For example, a pegylated interferon-α can be administered once every 2 to 8 weeks together with a daily dose of a BCR-ABL1 kinase inhibitor for a treatment period.

In a sequential treatment, during any given treatment period, multiple doses of a pegylated interferon-α or a BCR-ABL1 kinase inhibitor are first administered alone to a subject followed by multiple doses of a BCR-ABL1 kinase inhibitor or multiple doses of a pegylated interferon-α administered alone, respectively. For example, a subject can be given only a BCR-ABL1 kinase inhibitor daily for a time and then only a pegylated interferon-α once every 2 to 8 weeks for a time.

The pegylated interferon-α used in any of the methods described herein can be a conjugate of formula I:

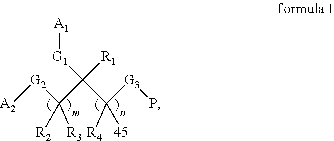

wherein each of R₁, R₂, R₃, R₄, and R₅ independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl; each of A₁ and A₂, independently, is a polymer moiety; each of G₁, G₂, and G₃, independently, is a bond or a linking functional group; P is an interferon-α moiety; m is 0 or an integer of 1-10; and n is an integer of 1-10. The conjugate of formula I is also described in WO2009/023826A1.

Referring to the above formula, the conjugate can have one or more of the following features: G3 is a bond and P is an interferon-α moiety (e.g., a human interferon-α-2b) in which the amino group at the N-terminus is attached to G3; A₁ and A₂ are polyalkylene oxide moieties having a molecular weight of 2-100 kD (preferably 10-30 kD), each of G₁ and G₂ is

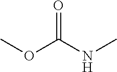

(in which O is attached to A₁ or A₂, and NH is attached to a carbon atom as shown in formula I), or each of G₁ and G₂ is urea, sulfonamide, or amide, (in which N is attached to a carbon atom as shown in formula I); m is 4, n is 2, and each of R₁, R₂, R₃, R₄, and R₅ is H; and the interferon-α moiety is a modified interferon-α moiety containing 1-4 additional amino acid residues. In some embodiments, the interferon-α moiety is a human interferon α-2b having an extra proline residue at the N-terminus and is 166 amino acids in length.

The conjugate can also have one or more of the following properties: (i) a median Tmax in the range of 3 to 6 days following administration of multiple 50 to 540 μg doses of the conjugate once every two weeks to subjects; (ii) a mean T_{1/2} in the range of 6 to 10 days following administration of multiple 50 to 540 μg doses of the conjugate once every two weeks to subjects; and (iii) an individual maximum tolerated dose of at least 500 μg once every 2 to 4 weeks in subjects.

In some embodiments, the conjugate is ropeginterferon alfa-2b, which has a predominant isoform having the formula:

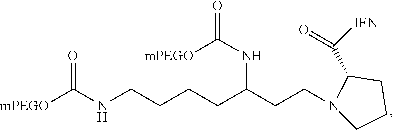

in which mPEG has a molecular weight of 20 kD and IFN is an interferon-α-2b (e.g., a human interferon-α-2b).

Ropeginterferon alfa-2b is produced by covalent attachment of a 40 kDa PEG molecule to the N-terminal proline residue of a Proline-Interferon-2b (Pro-IFN alpha-2b). Proline-interferon alpha-2b is generated by recombinant DNA technology introducing an extra proline residue to a human interferon α-2b at the N-terminus, giving a polypeptide of total 166 amino acids in length. Pro-IFN alpha-2b has a molecular weight of approximately 19 kDa and has the amino acid sequence identical to the theoretical sequence predicted excluding the additional N-terminal proline. It is then PEGylated with an approximately 40 kDa PEG moiety forming approximately 60 kDa PEGylated proline-interferon alpha-2b or ropeginterferon alfa-2b. The biological activity of ropeginterferon alfa-2b is determined by cytopathic effect (CPE)-based antiviral assay.

In any of the methods described herein, the pegylated interferon-α can be administered by any means known in the art, e.g., via subcutaneous or intravenous route. The pegylated interferon-α can be formulated as an injectable formulation. For example, it can be in the form of a ready-to-use prefilled syringe (PFS) containing, e.g., 0.2 to 2 mL of solution, that can be for self-injection. Each PFS can contain the labeled amount of the drug product, sodium chloride, sodium acetate anhydrous, acetic acid, benzyl alcohol, and polysorbate 80. The vehicle for the drug product can be sterile water for injection, and the drug product solution can have a pH of about 6.0.

The pegylated interferon-α can be administered, either sequentially or simultaneously with a BCR-ABL tyrosine kinase inhibitor, to a subject in need thereof for a time period (i.e., a treatment period) at a dose of 50 to 540 μg at a regular interval, which is at least 2 weeks, e.g., at least 2, 3, 4, 5, 6, 7, 8, or more weeks. For example, a dose can be administered every 2, 3, 4, 5, 6, 7, or 8 weeks. An interval that is defined in days or months is also contemplated. A regular interval of 10 to 60 days (e.g., 14, 21, 25, 26, 27, 28, 29, 30, 31, 35, 42, 49, and 56 days), one month, or two months can be utilized in the method.

The term “dose” refers to the amount of a compound administered to a subject at one time.

The term “interval” refers to the time between administration of two consecutive doses. In any of the methods described herein, the pegylated interferon-α is administered at an interval of 2 to 8 weeks, e.g., 2, 3, 4, 5, 6, 7, or 8 weeks. For example, a dose can be administered once every 2, 3, 4, 5, 6, 7, or 8 weeks. An interval that is defined in days or months is also contemplated. A regular interval of 10 to 60 days (e.g., 14, 21, 25, 26, 27, 28, 29, 30, 31, 35, 42, 49, and 56 days), one month, or two months can be utilized in any of the methods.

A treatment period can be at least 1, 2, 3, 4, 5, 6, 7, 8, 9, 10, 11, 12, 24, 36, 42, 48, 54, 60, 66, 72, 78, 84 or more months. In some embodiments, the treatment period is 1, 2, 3, 4, 4.5, 5, 5.5, 6, 6.5, 7, 7.5, 8, 8.5, 9, 9.5, 10 or more years.

A dose administered during a treatment period ranges from 50 to 540 μg. The dose can be 50 μg, up to 55 μg, specifically up to 60 μg, specifically up to 65 μg , specifically up to 75 μg, specifically up to 80 μg, specifically up to 85 μg, specifically up to 90 μg, specifically up to 95 μg, specifically up to 100 μg, specifically up to 105 μg, specifically up to 110 μg, specifically up to 115 μg, specifically up to 120 μg, specifically up to 125 μg, specifically up to 130 μg, specifically up to 135 μg, specifically up to 140 μg, specifically up to 145 μg, specifically up to 150 μg, specifically up to 155 μg, specifically up to 160 μg, specifically up to 165 μg, specifically up to 170 μg, specifically up to 175 μg, specifically up to 180 μg, specifically up to 185 μg, specifically up to 190 μg, specifically up to 195 μg, specifically up to 200 μg, specifically up to 205 μg, specifically up to 210 μg, specifically up to 215 μg, specifically up to 225 μg, specifically up to 230 μg, specifically up to 235 μg, specifically up to 240 μg, specifically up to 245 μg, specifically up to 250 μg, specifically up to 255 μg, specifically up to 260 μg, specifically up to 265 μg, specifically up to 270 μg, specifically up to 275 μg. specifically up to 280 μg, specifically up to 285 μg, specifically up to 290 μg, specifically up to 295 μg, specifically up to 300 μg, specifically up to 305 μg, specifically up to 310 μg, specifically up to 315 μg, specifically up to 320 μg, specifically up to 325 μg, specifically up to 330 μg, specifically up to 335 μg, specifically up to 340 μg, specifically up to 345 μg, specifically up to 350 μg, specifically up to 400 μg, specifically up to 450 μg, specifically up to 500 μg, or specifically up to 540 μg.

During any treatment period, the pegylated interferon-α can be administered at a constant dose, meaning that the same dose is administered each time or only minimally different doses are administered (e.g., dose variation or deviation of less than 10%, specifically less than 5%, specifically less than 1%). Alternatively, different doses can be administered at a regular interval during a treatment period. For example, the interferon can be administered at a particular dose at a regular interval for a certain time, and it can then be administered at a different dose (higher or lower than the first dose) at the same regular interval.

The subject can be a subject who has not been treated with an interferon before or a subject who had previously been administered a dose (e.g., 12.5, 15, 18.75, or 25 μg) of a type I interferon once per week or every two weeks. The subject can be a subject who has been treated previously with a therapy other than an interferon (e.g., HU).

A BCR-ABL1 kinase inhibitor used in any of the methods described herein can be imatinib, dasatinib, nilotinib, bosutinib, or ponatinib. The kinase inhibitor can be administered at 10 mg to 800 mg daily, e.g., 50 mg to 100 mg, 100 mg to 150 mg, 150 mg to 400 mg, 200 mg to 600 mg, 400 mg to 800 mg, up to 30 mg, up to 40 mg, up to 45 mg, up to 50 mg, up to 60 mg, up to 100 mg, up to 150 mg, up to 200 mg, up to 300 mg, up to 400 mg, up to 500 mg, up to 600 mg, up to 700 mg, or up to 800 mg, sequentially or simultaneously with a pegylated interferon-α, for a treatment period. The kinase inhibitor can be administered by any suitable route, e.g., orally as a pill or tablet.

During any treatment period, the BCR-ABL1 kinase inhibitor can be administered at a constant dose, meaning that the same dose is administered each time or only minimally different doses are administered (e.g., dose variation or deviation of less than 10%, specifically less than 5%, specifically less than 1%). Alternatively, different doses can be administered during a treatment period. For example, the kinase inhibitor can be administered at a specific daily dose then at one or more different daily doses during the treatment period.

The subject can be a subject who has not been treated with any BCR-ABL1 kinase inhibitor or a subject who had previously been administered a different BCR-ABL1 kinase. The subject can be a subject who has been treated previously with a therapy other than a BCR-ABL1 kinase (e.g., an interferon).

In any of the methods or treatment periods described herein, either or both of the pegylated interferon-α and BCR-ABL1 kinase inhibitor may be titrated. A subject can be treated with a lower starting dose of either drug. If the subject responds well (e.g., lack of significant drug-related adverse events, significant self-reported discomfort, abnormal hematological responses or other symptoms) after a time, the dose given to the subject may be increased incrementally. After that, the target dose is maintained during the treatment period. The dose can be increased successively until the desired dose is reached. For example, if the pegylated interferon-α is administered once every 2, 3, 4, 5, 6, 7, or 8 weeks, the dose can be increased every 2, 3, 4, 5, 6, 7 or 8 weeks, respectively. In some embodiments, the target dose is reached in 2 to 48 weeks from the first administration of the drug (e.g., 2 to 8 weeks, 4 to 12 weeks, 4 to 16 weeks, 4 to 20 weeks, 4 to 24 weeks, 6 to 12 weeks, 6 to 16 weeks, 6 to 20 weeks, 6 to 24 weeks, 6 to 28 weeks, 6 to 32 weeks, 6 to 36 weeks, 6 to 40 weeks, 8 to 12 weeks, 8 to 16 weeks, 8 to 20 weeks, 8 to 24 weeks, 8 to 28 weeks, 8 to 32 weeks, 8 to 36 weeks, 8 to 40 weeks, 12 to 16 weeks, 12 to 20 weeks, 12 to 24 weeks, 12 to 28 weeks, 12 to 32 weeks, 12 to 36 weeks, 12 to 40 weeks, or 12 to 48 weeks). During the titration process, any dose, prior to reaching the target dose, may be maintained for a time period (e.g., 2 to 16 weeks) or a number of successive doses (e.g., 2 to 30 successive doses) or reduced depending on the subject's response.

An initial dose or starting dose of a drug refers to the first dose administered to a subject during a treatment period (i.e., week 0). Prior to the treatment period, the subject can be naïve to interferon treatment or BCR-ABL1 kinase inhibitor treatment or may not have been administered the same pegylated interferon-α or kinase inhibitor.

A subjected treated with a pegylated interferon-α and a BCR-ABL1 kinase inhibitor in a method described herein, either simultaneously or sequentially, can have one or more of a molecular response, a hematologic response, and a cytogenetic response during or by the end of a treatment period. In some embodiments, one or more responses are detected by at least 3, 6, 9, 12, 15, 18, 21, 24, 27, 30, 33, or 36 months of treatment.

A molecular response or molecular remission is a reduction of BCR-ABL1 transcripts to a particular level according to the international scale (IS). For example, a molecular response can be a reduction of BCR-ABL1 transcripts to ≤0.1% or deeper, ≤0.01% or deeper, ≤0.0032% or deeper, ≤0.001% or deeper, or a non-detectable level. Quantitative methods of determining BCR ABL1 transcript levels in blood or bone marrow samples are known in the art.

A complete hematologic response or hematologic remission (CHR) is achieved when blood counts are normalized without immature cells and without splenomegaly. For example, a CHR can include one ore more of the following criteria: (1) white blood cell count <10×10⁹/L; (2) platelet count <450×10⁹/L; (3) absence of immature granulocytes (such as blasts, promyelocytes, and myelocytes) in peripheral blood; (4) less than 5% basophils in peripheral blood; (5) absence of extramedullary involvement; and (6) absence of splenomegaly.

A cytogenic response or cytogenic remission is determined by evaluation of percentages of cells containing the Philadelphia (Ph) chromosome in bone marrow samples. At least 20 dividing cells (metaphases) should be analyzed. The presence of greater than 95% Ph+ cells can be considered as a non-response. A partial cytogenic response can be 1% to 35% Ph+ cells, e.g., 5% or less, 10% or less, 15% or less, 20% or less, 25% or less, 30% or less, or 35% or less. A complete cytogenic response (CCyR) is defined as 0% Ph+ cells. A subject treated with a conjugate of formula I and a BCR-ABL1 kinase inhibitor, either simultaneously or sequentially, can exhibit less frequent adverse events (e.g., 5% to 100%, 10% to 30%, 20% to 40%, 30% to 50%, 40% to 60%, or 50% to 70% less of total adverse events, any adverse events, ≥grade 3 events, or ≥grade 4 events) or lower grade events (e.g., absence of ≥grade 3 events) than a subject treated with a different pegylated interferon and a BCR-ABL1 kinase inhibitor. The different pegylated interferon can be peginterferon alfa-2b or peginterferon alfa-2a administered weekly.

Adverse events can include hematologic, non-hematologic, or biochemical adverse events. Hematologic adverse events can include anemia, neutropenia, lymphopenia, thrombocytopenia, and pancytopenia. Non-hematologic adverse events can include infections, psychiatric disorders (e.g., depression), asthenia, fatigue, musculoskeletal pain, muscle cramps, abdominal pain, edema, dizziness, rash, headache, nausea, thrombosis, weight gain, weight loss, seizures, hemorrhage, diarrhea, or vomiting. Biochemical events can include elevated aspartate aminotransferase, alanine aminotransferase, or gamma-glutamyltransferase levels. Adverse events are graded based on standards accepted in the field (e.g., National Cancer Institute Common Terminology Criteria for Adverse Events).

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications cited herein are herein incorporated by reference in their entirety.

### EXAMPLE 1: METHODS

One objective of this phase 1 study was to determine the safety and tolerability of the addition of ropeginterferon alfa-2b to an established dose of imatinib in patients with chronic phase CML not having achieved a deep molecular remission (DMR) defined as a molecular response (MR) 4.5 or undetectable BCR-ABL1 transcripts. Another objective was to determine the rate of achieving an MR below 4.5 or undetectable BCR ABL1 transcripts.

The study was approved by the local review board. Five Austrian centers participated in this trial. All patients enrolled in the study gave written informed consent.

For study inclusion, patients required a diagnosis of CML in first chronic phase treated with imatinib as first-line therapy and a complete hematologic remission (CHR) as well as a complete cytogenetic remission (CCyR) after at least 18 months of treatment. Other inclusion criteria were as follows: adequate organ function (total bilirubin<1.5×upper limit of norm [ULN], aspartate aminotransferase and alanine aminotransferase<2.5×ULN, creatinine<1.5×ULN, absolute neutrophil count>1.5×10⁹/L, and platelets>100×10⁹/L), and Eastern Cooperative Oncology Group (ECOG) performance status<3.

Patients were excluded if they had a MR 4.5 or undetectable BCR-ABL1 transcripts measured at a central laboratory. Other exclusion criteria were severe or uncontrolled concomitant organ disease (e.g., respiratory, cardiac, hepatic, or renal disease), autoimmune disease (e.g., connective tissue disease, rheumatoid arthritis, immune thrombocytopenia, autoimmune thyroiditis, psoriasis, lupus nephritis, or any other autoimmune disorder), human immunodeficiency virus infection, and any other active or chronic infectious disease. Patients with prior malignancies were required to be disease free for at least 5 years (except treated basal cell or squamous cell carcinoma of the skin, and “in situ” carcinoma of the cervix or breast). Pregnant or breast-feeding women were excluded from study entry as well.

Imatinib was continued at the same dose administered before study entry. Ropeginterferon alfa-2b given subcutaneously was added at a dose of 50 μg every 14 days for 12 weeks. In the absence of dose-limiting toxicities (DLTs), ropeginterferon alfa-2b was increased to 100 μg every 14 days for another 12 weeks, and if tolerated, continued for 12 more months. The duration of treatment was limited to a maximum of 18 months. DLT was defined as hematologic or non-hematologic toxicities≥grade 3.

In the case of occurrence of a DLT with ropeginterferon alfa-2b at 50 μg every 2 weeks, the study drug was to be withheld until the toxicity had resolved to ≤grade 1, then reintroduced at a dose of 50 μg every 14 days. If a second DLT occurred at the same dose, ropeginterferon alfa-2b was to be stopped and study participation discontinued. In the absence of another DLT after reintroduction of ropeginterferon alfa-2b at a dose of 50 μg every 2 weeks, the dose was then increased to 100 μg every 2 weeks after 12 weeks. If a DLT occurred at a dose of 100 μg, therapy had to be stopped until the toxicity had resolved to ≤grade 1. Treatment was then reintroduced with 50 μg every 2 weeks and after 12 weeks increased to 100 μg every 2 weeks if no DLT was observed. AEs were graded using the National Cancer Institute Common Terminology Criteria for Adverse Events (NCI CTCAE) version 4.0.

Real-time polymerase chain reaction-based monitoring of BCR-ABL1 transcripts was carried out using a European standardized approach established by a European collaborative group in two central laboratories. See, Gabert et al., Leukemia (2003), 17(12):2318-2357; and Mueller et al., Leukemia (2009), 23(11):1957-1963. BCR-ABL1 quantification was done every 12 weeks and at the final visit.

MMR was defined as a reduction of BCR-ABL1 transcripts to ≤0.1% according to International Scale (IS). A reduction of BCR-ABL transcripts to ≤0.01% was defined as MR 4, a reduction to ≤0.0032% as MR 4.5, and to ≤0.001% as MR 5. In this protocol, a DMR was defined as MR 4.5 or non-detectable BCR-ABL1 transcripts.

The intended-to-treat population included all enrolled patients who received at least one dose of study treatment and was used for all analyses. Patients who discontinued their participation in the study due to AEs or toxicity prior to the key response evaluation were considered as treatment failures. Baseline characteristics were summarized for all patients enrolled using appropriate descriptive statistics, i.e., number (%) of patients for categorical variables and mean, median, minimum/maximum, and interquartile range (IQR) for continuous variables. The primary efficacy parameter was the achievement of a MR defined as 4.5 or deeper. Wilcoxon and McNemare tests were used to compare BCR-ABL1 transcript levels and MR categories at 12 and 18 months versus screening time point. The assessment of safety was mainly based on the frequency of AEs, particularly those leading to discontinuation of treatment and on the number of significant laboratory abnormalities. AEs were coded using MedDRA dictionary version 21.1 and were summarized by presenting the number and percentage (as appropriate) of patients having any AE by body system, type of AE, and maximum severity.

### EXAMPLE 2: RESULTS

### Patient Characteristics

12 patients (11 males and one female) were enrolled. See Table 1. Nine patients (75%) completed the study treatment according to protocol. Three patients terminated the study treatment early, one due to occurrence of a DLT (neutropenia grade 3), one due to an AE (panic attacks grade 2) and one due to the patient's decision.

TABLE 1
Patient characteristics

| Baseline characteristics | N = 12 | |
|---|---|---|
| Age, years | 39 | (32-69, 35-49) |
| Male | 11 | (92%) |
| ECOG performance status zero | 12 | (100%) |
| Leucocytes, x10⁹ cells/L | 4.8 | (3.6-8.6, 4.3-6.1) |
| Platelets, x10⁹ cells/L | 186.5 | (124-291, 161-208.8) |
| Hemoglobin, g/dL | 14.3 | (12.3-16.7, 13.1-14.6) |
| Thyroid stimulating hormone, mU/L | 1.14 | (0.06-2.06, 0.72-1.44) |
| Antinuclear antibodies negative | 11 | (92%) |
| Aspartate aminotransferase, U/L | 28.5 | (20-42, 25.3-35.8) |
| Alanine aminotransferase, U/L | 27 | (14-87, 25-37.5) |
| Gamma-glutamyltransferase, U/L | 25 | (13-56, 17.8-31.3) |
| CHR at study entry | 12 | (100%) |
| CCyR/MR 3.0 at study entry | 12 | (100%) |
| Imatinib before study entry, months | 37 | (20-131, 26-77) |

Note:
Data are n (%), or median (range, IQR).
Abbreviations: CCyR, complete cytogenetic remission; CHR, complete hematologic remission; ECOG, Eastern Cooperative Oncology Group; MR, molecular remission.

Median age was 39 years (range 32-69, IQR 35-49). All patients had an ECOG performance status of 0. At study entry all patients had a CHR as well as a CCyR and/or an MR 3. Median dose of imatinib at study entry was 400 mg (range 400-800 mg). The mean dose of ropeginterferon alfa-2b was 70 μg every 2 weeks (range 45-92 μg). The median number of ropeginterferon alfa-2b cycles was 38.5 (range 1-39; IQR 30-39).

Median duration of imatinib treatment before study entry was 37 months (range 20-131; IQR 26-77).

### Safety

All 12 patients who received at least one dose of study drug were assessed for toxicity. Most of the non-hematological AEs reported were grade 1/2. Hematological AEs was mainly grade 2 neutropenias with grade 3 neutropenias reported in three patients. See Table 2. All non-hematological AEs were known AEs of IFN or imatinib. No new AEs were reported.

TABLE 2
Adverse events

| Number of patients with | | |
|---|---|---|
| maximum grade (N = 12) | | |
| Grade 1 | Grade 2 | Grade 3 |

Adverse events (excerpt)

| | | | | | |
|---|---|---|---|---|---|
| Infections^{a} | 0 | 7 | (58%) | 1 | (8%) |

| Psychiatric disorders^{a} | 1 | (8%) | 2 | (17%) | 0 |
|---|---|---|---|---|---|
| Asthenia | 1 | (8%) | 0 | | 0 |
| Fatigue^{b} | 5 | (42%) | 0 | | 0 |
| Musculoskeletal pain^{b} | 3 | (25%) | 0 | | 0 |
| Dizziness | 2 | (17%) | 0 | | 0 |
| Headache | 5 | (42%) | 0 | | 0 |
| Rash^{b} | 3 | (25%) | 1 | (8%) | 0 |

Laboratory

| Leukopenia | 3 | (25%) | 7 | (58%) | 0 | |
|---|---|---|---|---|---|---|
| Neutropenia | 1 | (8%) | 6 | (50%) | 3 | (25%) |
| Thrombocytopenia | 9 | (75%) | 0 | | 0 | |
| Anemia | 7 | (58%) | 0 | | 0 | |
| ASAT elevated | 7 | (58%) | 0 | | 1 | (8%) |
| ALAT elevated | 5 | (42%) | 1 | (8%) | 1 | (8%) |
| GGT elevated | 4 | (33%) | 1 | (8%) | 1 | (8%) |

Abbreviations: AE, adverse event; ALAT, alanine-aminotransferase; ASAT, aspartate-aminotransferase; GGT, gamma-glutamyltransferase.
^{a}MedDRA System Organ Class.
^{b}Adverse event terms are summarized MedDRA preferred terms; no events grade 4 or 5.

Treatment had to be discontinued in three out of 12 patients (25%). One patient experienced a recurrent grade 3 neutropenia (DLT). One patient suffered from panic attacks of grade 2 severity after one dose of ropeginterferon alfa-2b and refused further treatment with the study drug, and another patient withdrew for personal reasons. Discontinuation rates in two other randomized studies comparing imatinib with combination of imatinib with other pegylated IFN-2a or -2b, the French SPIRIT study and the trial conducted by the Nordic group, were clearly higher with 45% and 60% in the combination arms, respectively. See, Preudhomme et al., N Engl J Med. (2010), 363(26):2511-2521; and Simonsson et al., Blood (2011), 118(12):3228-3235. The French SPIRIT trial tested combining imatinib 400 mg daily with peginterferon alfa-2a at 90 μg per week. The Nordic CML study group investigated imatinib in combination with peginterferon alfa-2b.

### Response

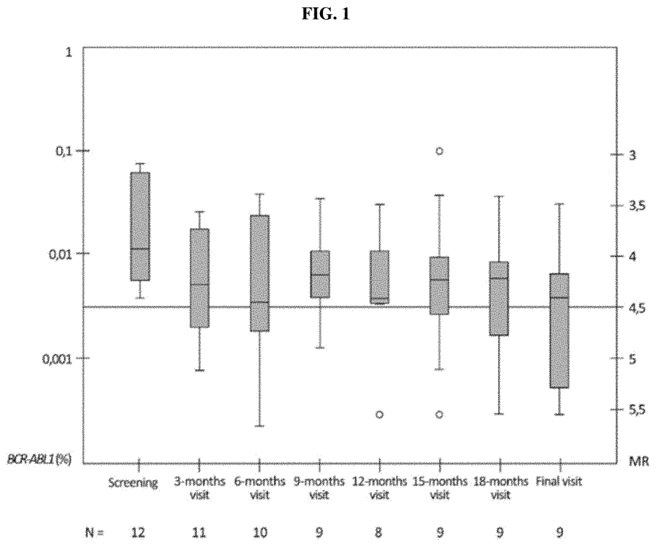

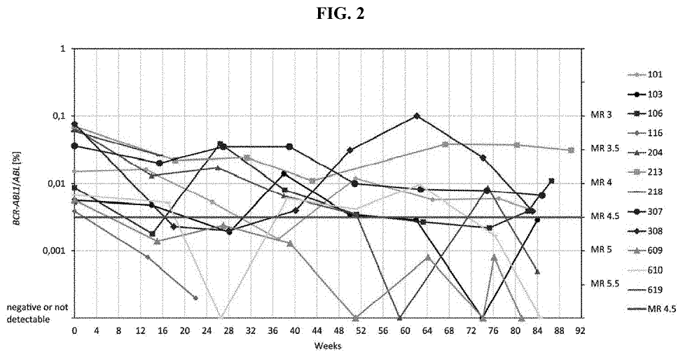

BCR ABL1 transcript levels were significantly lower at 12 months as well as at the final visit compared to the screening visit (p=0.012. See FIGS. 1 and 2; and Table 3. No significant difference of the BCR-ABL1 transcript level was found between 12 and 18 months of ropeginterferon alfa-2b treatment (p=0.128). One patient had an MR 4.5 at 12 months, while four patients each had an MR 4.5 at 18 months, which was statistically significant (p=0.0125).

TABLE 3
BCR-ABL1 evaluation
Number of patients with decrease in BCR-ABL1

| | | |
|---|---|---|
| After 12 months | 8/8^{a} | (100%) |
| At final visit (1 month after last | 8/9 | (88.9%) |
| ropeginterferon alpha-2b) | | |
| After 12 months to final visit | 7^{b}/8^{a} | (87.5%) |

Number of patients with molecular remission <4.5

| | | |
|---|---|---|
| After 12 months | 1/8^{a} | (12.5%) |
| At final visit (1 month after last | 4/9 | (44.4%) |
| ropeginterferon alpha-2b) | | |

^{a}One patient was not evaluable after 12 months.
^{b}One patient was negative after 12 months and at final visit.

To determine whether a DMR was associated with the cumulative dose of ropeginterferon alfa-2b, the median cumulative IFN doses in patients with DMR versus without a DMR were compared. Patients achieving a DMR were treated with lower cumulative doses of ropeginterferon alfa-2b compared to patients not achieving a DMR (p=0.138). Also ropeginterferon alfa-2b cumulative doses administered during the first 12 months showed no significant association with the achievement of a DMR (all p values >0.05).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

What is claimed is:

1. A method of treating chronic myeloid leukemia in a subject, the method comprising administering to a subject in need thereof a pegylated interferon-α and a BCR-ABL tyrosine kinase inhibitor simultaneously or sequentially for a treatment period, wherein the pegylated interferon-α is a conjugate of formula I: 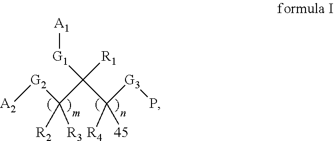
in which
each of R₁, R₂, R₃, R₄, and R₅, independently, is H, C₁₋₅ alkyl, C₂₋₅ alkenyl, C₂₋₅ alkynyl, aryl, heteraryl, C₃₋₈ cycloalkyl, or C₃₋₈ heterocycloalkyl;
each of A₁ and A₂, independently, is a polymer moiety;
each of G₁, G₂, and G₃, independently, is a bond or a linking functional group;
P is an interferon-α moiety;
m is 0 or an integer of 1-10; and
n is an integer of 1-10.

2. The method of claim 1, wherein the conjugate has one or more properties including:
(i) a median Tmax in the range of 3 to 6 days following administration of multiple 50 to 540 μg doses of the conjugate once every two weeks to subjects;
(ii) a mean T_{1/2} in the range of 6 to 10 days following administration of multiple 50 to 540 μg doses of the conjugate once every two weeks to subjects; and
(iii) an individual maximum tolerated dose of at least 500 μg once every 2 to 4 weeks in subjects.

3. The method of claim 1, wherein the conjugate has one or more features including: G3 is a bond and P is an interferon-α moiety in which the amino group at the N-terminus is attached to G3; A₁ and A₂ are polyalkylene oxide moieties each having a molecular weight of 10-30 kD; each of G₁ and G₂ is 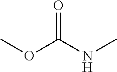
in which O is attached to A₁ or A₂, and NH is attached to a carbon atom as shown in formula I; each of R₁, R₂, R₃, R₄, and R₅ is H; m is 4 and n is 2; and the interferon-α moiety is a modified interferon-α moiety containing 1-4 additional amino acid residues.

4. The method of claim 1, wherein the interferon-α moiety is a human interferon-α_{2b} having a proline residue at the N-terminus and is 166 amino acids in length.

5. The method of claim 1, wherein the conjugate is 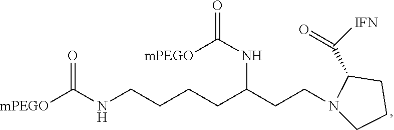
in which mPEG has a molecular weight of 20 kD and IFN is an interferon-α_{2b}.

6. The method of claim 1, wherein the tyrosine kinase inhibitor is imatinib, dasatinib, nilotinib, bosutinib, or ponatinib.

7. The method of claim 6, wherein 100 to 800 mg of imatinib is administered to the subject daily.

8. The method of claim 1, wherein 50 to 540 μg of the pegylated interferon-α is administered to the subject once every 2 to 8 weeks.

9. The method of claim 8, wherein 50 to 100 μg of the pegylated interferon is administered to the subject once every 2 weeks.

10. The method of claim 1, wherein the treatment period is at least 6 months, at least 12 months, at least 18 months, at least 24 months, at least 30 months, at least 36 months, at least 42 months, at least 48 months, or at least 54 months or more.

11. The method of claim 1, wherein the subject was previously treated with imatinib or another BCR-ABL tyrosine kinase inhibitor alone, or an interferon or pegylated interferon alone.

12. The method of claim 1, wherein the subject is treated with the pegylated interferon-α and the BCR-ABL tyrosine kinase inhibitor sequentially.

13. The method of claim 12, wherein the subject is first treated with the BCR-ABL tyrosine kinase inhibitor alone followed by the pegylated interferon-α alone, or is first treated with the pegylated interferon-α alone followed by the BCR-ABL tyrosine kinase inhibitor alone.

14. The method of claim 1 wherein the subject has a reduction of BCR-ABL1 transcript to at least ≤0.01% or deeper during or by end of the treatment period.

15. The method of claim 14, wherein the subject has a reduction of BCR-ABL1 transcript to at least ≤0.0032% or deeper during or by the end of the treatment period.

16. The method of claim 14, wherein the reduction is detected at 12 months or earlier in the treatment period.

17. The method of claim 1, wherein the subject has a complete hematologic response or a cytogenetic response or both during or by end of the treatment period.

18. The method of claim 17, wherein the subject has a complete cytogenic response.

19. The method of claim 18, wherein the subject has a complete hematologic response and a complete cytogenetic response.

20. The method of claim 1, wherein the subject has less adverse events or lower grade adverse events than a subject treated with a combination of a BCR-ABL tyrosine kinase inhibitor and another pegylated interferon.
